(19) United States
(12) Patent Application Publication
(10) Pub. No.: US 2023/0017186 A1
(43) Pub. Date: Jan. 19, 2023

(54) Systems and Methods for Measuring Concentration of an Analyte

(71) Applicant: Brolis Sensor Technology, UAB, Vilnius (LT)

(72) Inventors: Ieva Simonyte, Vilnius (LT); Augustinas Vizbaras, Vilnius (LT); Tadas Buciunas, Panevezys (LT); Arunas Miasojedovas, Vilnius (LT); Stephan Heinz Sprengel, Vilnius (LT)

(73) Assignee: Brolis Sensor Technology, UAB, Vilnius (LT)

(21) Appl. No.: 17/782,239

(22) PCT Filed: Dec. 3, 2020
(86) PCT No.: PCT/EP2020/084552

§ 371 Date: Jun. 3, 2022

Related U.S. Application Data
(60) Provisional application No. 62/944,644, filed on Dec. 6, 2019.

(51) Int. Cl. G01N 21/39; G01N 21/27

(52) U.S. Cl. CPC G01N 21/39; G01N 21/274; A61B 5/1455

(57) ABSTRACT

Techniques for acquiring and processing data in combination with a photonic sensor system-on-a-chip (SoC) (1) to provide real-time calibrated concentration levels of an analyte (e.g., a constituent molecule within a biological substance) are described. A raw signal (1300) to be analyzed is collected by the sensor chip (1) via diffuse reflectance or transmittance. Determination of the analyte concentration is based on, in part, Beer-Lambert principles and facilitated by applying (2240) scattering correction to the raw signal (1300) prior to decomposition and analysis thereof.

### CROSS-REFERENCE TO RELATED APPLICATIONS

This application claims priority to and benefit of U.S. Provisional Patent Application No. 62/944,644, entitled “Systems and Methods for Measuring Concentration of an Analyte,” filed on Dec. 6, 2019, the entire contents of which are incorporated herein by reference.

### FIELD OF THE INVENTION

Embodiments of this invention relate to a method of data acquisition from a target biological substance by optical communication between the target substance and the III-V/IV semiconductor photonic sensor and the method of data processing to retrieve an absolute concentration level of a target molecule within the substance. This is applicable but not limited to transdermal sensing and monitoring of blood glucose, urea, lactate, creatinine, ethanol and other constituent molecules by means of tunable wavelength absorption spectroscopic sensing. The described technology is compatible with consumer electronics technology platforms in terms of manufacturing technology and size, weight, power, and cost requirements and offers a pivotal advantage in terms of usefulness for wearable healthcare device technology. This technology may be utilized by people impaired by chronic diseases such as diabetes where currently no non-invasive sensing solution exist. Moreover, a novel approach is provided for continuously monitoring vital physiological markers non-invasively, where currently only a point-of-care solution exists.

### BACKGROUND

Many techniques for spectroscopic, non-invasive measurement of analytes, such as measurement of blood glucose using near-infrared spectroscopy, employ a broadband light source, such as halogen lamp. The electromagnetic radiation (EMR) emitted from such a source, and that received from a medium to be analyzed (e.g., diffusively reflected by or transmitted through the medium), have components at a number of wavelengths. The components from the EMR received from the medium are typically separated using a grating technique to obtain a spectrum. A spectrometer having a broadband source and a grating mechanism is typically a large, complex structure, that can be cumbersome or impractical for in-field or at-home use.

Photonic systems-on-a-chip (P-SoC) offer ultimate size reduction potential which is necessary for large volume applications such as consumer electronics markets, automotive, home-use medical devices, etc. The P-SoC concept combines all or most of the functions of a general photonic system and enables those functions to be realized within a single chip assembly. Typically, this can be realized as a monolithic photonic integrated circuit (PIC) based on III-V semiconductor or a combination of III-V semiconductor and group-IV semiconductor. The first approach allows all active and passive optical components be realized within the same wafer, allowing a completely monolithic device. This is ideal as all light sources and detectors are inherently aligned to the waveguides and do not require any assembly steps. However, inherent III-V material properties, such as higher absorption and lower light confinement in the waveguides and thus larger waveguides bending radii to reduce bend loss, together with complex technology requiring multiple epitaxial growths, limit the scaling potential to very large markets such as consumer electronics, as very low cost per chip is required by the market. As a trade-of, hybrid III-V/IV P-SoC offer a solution where light generation function is realized within the III-V semiconductor chip and light routing, filtering and other functions are realized within a group-IV semiconductor chip. Light detection depending on the wavelength of EMR, can be realized either within group-III-V or group-IV semiconductor chip. Hybrid approach proves to be beneficial for large volumes markets as group-IV semiconductor manufacturing technologies such as CMOS—for example offer unmatched scaling potential. Techniques for analyte measurement using P-SoC are generally not known, however.

### SUMMARY

Hybrid integration of III-V semiconductor chips with group-IV semiconductor photonic integrated circuit offers the potential to combine the best of two worlds, with light detection and light generation function being realized within direct bandgap III-V semiconductor for ultimate efficiency, performance, cost, and yield, whereas passive functions such as light filtering, routing, locking, feedback control are realized within a photonic integrated circuit (PIC) within a group-IV semiconductor, for example silicon-on-insulator, or silicon-on-silicon nitride, or silicon on silicon nitride or silicon on insulator. In various embodiments, a swept wavelength laser based photonic system on a chip with integrated emission wavelength tuning (sweeping), and wavelength shift tracking and absolute wavelength calibration functions, are deployed for remote acquisition of relevant data from a biological object such as a living object. In different embodiments, acquired data is then processed to provide biomolecule specific absolute value such as concentration level and/or concentration level as a function of time (trend). The combination of the hybrid III-V/IV semiconductor platform and techniques for processing the acquired data on-chip offers new opportunities for wearable device platforms such as for example, smart watches for monitoring important physiological parameters in real time.

Techniques for acquiring and processing data in combination with a photonic sensor system-on-a-chip to provide real-time calibrated concentration levels of an analyte (e.g., a constituent molecule within a biological substance) are described. The biological substance may be blood, interstitial fluid, tissue or a combination of substances. The photonic sensor system-on-a-chip (SoC) assembly includes a hybrid III-V and group-IV semiconductor assembly, with the III-V semiconductor elements providing optical gain and detection functions, and optical feedback, light routing, filtering, locking and other passive functions being provided within the group-IV semiconductor photonic integrated circuit.

In use, the assembly is in optical communication with the biological substance, and the sensor may be remote from the substance (in vivo scenario) or embedded within the substance (implanted). The sensor interacts with the target substance via optical communication, the light from the sensor interacts with the substance, and the light signal is modulated due to light-molecule interaction, where the interaction is molecule specific. After the interaction, the signal is collected by the sensor chip by means of diffuse reflectance or transmittance.

In practical scenarios, where such photonic sensor performs a direct transdermal measurement with a living object or is implanted within a living object, the raw signal collected by the sensor is very complex due to the complex nature of a typical biological substance, such as whole blood and or tissue. Various data analysis techniques described herein in combination with the hardware (e.g., the Soc) can be used to retrieve calibrated concentration level values from most complex biological substances. This is of particular importance for transdermal/implanted monitoring of vital metabolites such as glucose, lactate, urea, ethanol, serum albumin, creatinine, and others both for subjects impaired with chronic diseases such as diabetes, kidney or liver malfunctions as well as acute clinical cases such as sepsis or fitness level or diet monitoring for athletes and general public.

Accordingly, in one aspect a method is provided for calibrating a sensor for measurement of the concentration of an analyte in a medium. The method includes collecting, using a hybrid group III-V/group IV semiconductor photonics system-on-a-chip (SoC), a number of raw spectra from an object (e.g., the medium or sample) having the analyte. The method also includes partitioning the raw spectra according to respective spectral shapes thereof into a set of clusters, where each cluster includes a group of raw spectra. The method further includes, within each cluster: (i) applying a respective local scattering correction (LSC) to each raw spectrum belonging to the cluster to obtain a group of locally corrected spectra; and (ii) deriving a cluster-specific optimized set of pre-processing parameters and a cluster-specific calibration vector. The optimized set of pre-processing parameters and the calibration vector are derived using the locally corrected spectra and gold standard analyte concentration values corresponding to the group of raw spectra belonging to the cluster,

In some embodiments, deriving the cluster-specific optimized set of pre-processing parameters and the cluster-specific calibration vector for a particular cluster includes: (i) evaluating each one of a number of candidate sets of pre-processing parameters, where the evaluation of a particular candidate set includes: (A) pre-processing each locally corrected spectrum belonging to the particular cluster using the particular candidate set; (B) deriving a candidate calibration vector by applying multivariate regression calibration to the pre-processed locally corrected spectra and using the gold standard analyte concentration values corresponding to the group of raw spectra belonging to the particular cluster; and (C) computing a corresponding accuracy measure for the candidate calibration vector via cross-validation. Thereafter, the candidate set and the corresponding candidate calibration vector associated with a maximum accuracy measure are designated as the cluster-specific optimized set of pre-processing parameters and cluster-specific calibration vector, respectively.

The cluster-specific optimized set of pre-processing parameters may include a set of data processing parameters such as a) order of filtering, b) sort or type of filter used for smoothing, c) order of derivatives used for baseline removal, etc. The optimized set of parameters may be stored in the memory and may be used subsequently to preprocess data in the sensing mode.

The object may include tissue, and the analyte may include blood glucose, blood lactate, ethanol, creatinine, keratin, collagen, urea, serum albumin globulin, troponin, acetone, acetate, hydroxybutyrate, cholesterol, albumin, globulin, ketones-acetone, or water among others.

In some embodiments, the step of partitioning the raw spectra according to respective spectral shapes thereof includes applying a global scattering correction (GSC) to each of the raw spectra to obtain several globally corrected spectra. The partitioning step may also include clustering the several globally corrected spectra according to: (A) a specified number of clusters, (B) a specified maximum distance of a globally corrected spectrum from a centroid of a cluster, or (C) both the specified number of clusters and the specified maximum distance of a globally corrected spectrum from a centroid of a cluster. The partitioning step may further include, within each cluster, designating to that cluster a respective raw spectrum corresponding to a globally corrected spectrum belonging to the cluster. The clustering may include k-means clustering, affinity propagation, or agglomerative clustering.

In some embodiments, the method further includes storing in the SoC a GSC reference spectrum generated as part of the global scattering correction. The global scattering correction may be implemented as global multiplicative scattering correction, global standard normal variate (SNV) correction, global mean centering and normalization correction, Kubelka-Munk (K-M) correction, Saunderson correction, or a combination thereof. The local and/or global scattering correction may incorporate particle-size difference correction and/or pathlength-difference correction and may utilize K-M correction, Saunderson correction, multiplicative scattering correction, or a combination thereof. In some embodiments, the method includes storing in the SoC, for each cluster: (i) a corresponding LSC reference spectrum, and/or (ii) a corresponding calibration vector, (iii) cluster centroids, and/or (iv) the optimized set of pre-processing parameters for each cluster. The local scattering correction may also be implemented as local multiplicative scattering correction, or local standard normal variate (SNV) correction, local mean centering and normalization correction, K-M correction, Saunderson correction, or a combination of the aforementioned correction techniques, to achieve the linearization effect. Global and local scattering correction, when chosen appropriately, allow to account for particle-size difference effect on light scattering as well as account for optical path difference correction in tissue, e.g., to linearize the raw spectra, so that both linear Beer-Lambert absorption law as well as linear regression, including multivariate partial least square, techniques are applicable.

In some embodiments, determining the respective spectral shapes of the several raw spectra includes pre-processing the raw spectra by applying thereto a linear transformation and a baseline correction based on a reference spectrum of a selected analyte. The pre-processing may include Kubelka-Munk correction, Saunderson correction, multiplicative scattering correction, or a combination of any two or all three correction techniques.

In another aspect, a method is provided for measuring concentration of an analyte, where the method includes obtaining, using a hybrid group III-V/group IV semiconductor photonics system-on-a-chip (SoC), a raw spectrum from an object (e.g., a medium or a sample) having the analyte, and identifying from a number of clusters of spectra a cluster to which the raw spectrum belongs, where the cluster is identified based on the spectral shape of the raw spectrum. The method also includes applying a local scattering correction (LSC) to the raw spectrum to obtain a locally corrected spectrum, pre-processing the locally corrected spectrum using a cluster-specific optimized set of pre-processing parameters, and multiplying the pre-processed locally corrected spectrum with a cluster-specific calibration vector to obtain a corresponding calibrated concentration value for the analyte.

In some embodiments, obtaining the raw spectrum includes directing from the SoC to the object electromagnetic radiation (EMR) tunable at several different wavelengths, measuring using the SoC intensities of EMR received from the object at each of the different wavelengths, and converting the intensities into absorbance values, so that the raw spectrum includes an absorbance spectrum. The several different wavelengths may be selected from a range 1000-3500 nm or a range 1900-2500 nm.

In some embodiments, the clusters of spectra correspond to spectra collected previously using the SoC, and each of the clusters may be represented via a respective LSC reference, a respective cluster centroid, and/or a respective calibration vector, where the respective LSC reference, the respective cluster centroid, and the respective calibration vector for each cluster may be stored on the SoC. Identifying from the several clusters of spectra the cluster to which the raw spectrum belongs may include deriving a globally corrected spectrum using a global scattering correction (GSC) reference. Identification of the cluster to which the raw spectrum belongs may also include, within each of the several clusters, comparing the globally corrected spectrum with a respective LSC reference to obtain a distance corresponding to that cluster, and selecting a cluster for which the corresponding distance is minimum.

The global scattering correction may be implemented as global multiplicative scattering correction, global standard normal variate (SNV) correction, global mean centering and normalization correction, K-M correction, Saunderson correction, or a combination thereof. The local and/or global scattering correction may incorporate particle-size difference correction and/or pathlength-difference correction. The local scattering correction may be implemented as local multiplicative scattering correction, or local standard normal variate (SNV) correction, or local mean centering and normalization correction, K-M correction, Saunderson correction, or a combination thereof. LSC and GSC involve performing a linearizing transformation on the raw spectra to account for tissue/object scattering and absorption, to facilitate further data processing based on linear absorption techniques such as those based on the Beer-Lambert law, where the spectrum is decomposed into individual components and/or is processed further using PLS linear regression or a similar technique.

In some embodiments, determining the spectral shape of the raw spectrum includes pre-processing the raw spectrum by applying thereto a linear transformation and a baseline correction based on a reference spectrum of a selected analyte. The pre-processing may include Kubelka-Munk correction, Saunderson correction, multiplicative scattering correction, or a combination of any two or all three correction techniques.

In another aspect, a system for measuring concentration of an analyte includes a hybrid group III-V/group IV semiconductor photonics system-on-a-chip (SoC) for obtaining a raw spectrum from an object (e.g., a medium or a sample) having the analyte, and a processing unit that includes a processor and memory and that is configured to perform certain operations so as to measure the analyte concentration, store information, etc. Specifically, the processing unit is configured to obtain, using the hybrid group III-V/group IV semiconductor photonics system-on-a-chip (SoC), a raw spectrum from an object having the analyte, and to identify from a number of clusters of spectra a cluster to which the raw spectrum belongs, based on the spectral shape of the raw spectrum. The processing unit is also configured to apply a cluster-specific local scattering correction (LSC) to the raw spectrum, to obtain a locally corrected spectrum. The processing unit is further configured to pre-process the locally corrected spectrum using a cluster-specific optimized set of pre-processing parameters, and to multiply the pre-processed locally corrected spectrum with a cluster-specific calibration vector, to obtain a calibrated concentration value for the analyte.

In some embodiments, to obtain the raw spectrum, the SoC is configured to direct to the object electromagnetic radiation (EMR) that is tunable at several wavelengths, and to measure intensities of the EMR received from the object at each of the wavelengths. The processing unit is programmed to convert the intensities into absorbance values, so that the raw spectrum includes or is represented as an absorbance spectrum. The SoC may be configured to emit EMR at wavelengths in the range 1900-2500 nm or in the range 1000-3500 nm.

The several clusters of spectra may correspond to spectra collected previously using the SoC. Each of the clusters may represented via a respective LSC reference and a respective calibration vector. The SoC may include memory for storing, for each cluster, the respective LSC reference and the respective calibration vector, as well as a global scattering correction reference (also called a global scattering correction vector). The memory of the SoC may also store, for each cluster, the corresponding optimized set of pre-processing parameters.

In some embodiments, to identify among the several clusters of spectra the cluster to which the raw spectrum belongs, the processor is programmed to derive a globally corrected spectrum using a global scattering correction (GSC) reference stored in the memory. The processor may also be programmed to, within each cluster (i) compare the globally corrected spectrum with a respective LSC reference to obtain a distance corresponding to that cluster, and (ii) select a cluster for which the corresponding distance is minimum. The global scattering correction may include global multiplicative scattering correction, or global standard normal variate (SNV) correction, or global mean centering and normalization correction. Similarly, the local scattering correction may include local multiplicative scattering correction, or local standard normal variate (SNV) correction, or local mean centering and normalization correction. The local and/or global scattering correction may incorporate linearizing transformation for particle-size difference correction and/or pathlength-difference correction.

In some embodiments, the SoC includes a wavelength shift tracker to track a shift in wavelength of radiation emitted by the SoC, and/or a wavelength tracker to track absolute wavelength of the radiation emitted by the SoC, and/or a temperature sensor to measure temperature of the chip, and/or an SoC output power monitor to monitor or measure the intensity of the EMR emitted by the SoC during a wavelength sweep, so as to obtain a power curve.

In some embodiments, to determine the respective spectral shapes of the several raw spectra, the processor is configured to pre-processing the raw spectra by applying thereto a linear transformation and a baseline correction based on a reference spectrum of a selected analyte. In order to perform the pre-processing, the processor may be configured to apply Kubelka-Munk correction, Saunderson correction, multiplicative scattering correction, or a combination of any two or all three correction techniques.

### BRIEF DESCRIPTION OF THE DRAWINGS

FIG. 1 is a schematic block diagram of a photonic SoC deployed for remote sensing of an object, in accordance with an embodiment of the invention;

FIG. 2 is a simplified schematic diagram of the photonic sensor system deployed for a sensing experiment, in accordance with an embodiment of the invention;

FIG. 3 is a simplified schematic block diagram of the algorithm used in combination with the hardware illustrated in FIGS. 1 and 2 to create a calibration algorithm for a sensor, in accordance with an embodiment of the invention;

FIG. 4a is a graph of a large set of accumulated raw absorbance spectra from a piglet with a global MSC vector indicated in bold, and FIG. 4b is a graph of spectra after baseline correction (MSC) and before the clustering procedure, in accordance with an embodiment of the invention;

FIG. 5 is a graph of baseline corrected spectra from FIG. 4b, clustered into 6 different clusters (N=6 by definition) using k-means algorithm, with a maximum distance to the centroid within each cluster being indicated within the graph;

FIG. 6 is a block diagram illustrating algorithm schematics for construction of individual calibration models, in accordance with embodiments of the invention;

FIG. 7a is a graph of raw spectra collected from the object—a piglet—within one cluster before applying the MSC baseline correction. The bold black spectrum is the calculated local MSC reference, and FIG. 7b is a graph of spectra within the same cluster after baseline correction (MSC);

FIG. 8 is a graph of individual constituent concentration calibration vectors obtained for the glucose molecule using a transdermal diffuse reflectance sensing geometry with a piglet;

FIG. 9 is a schematic block diagram of the sensing algorithm used in combination with the hybrid III-V/IV semiconductor photonics sensor SoC, in accordance with an embodiment of the invention;

FIG. 10 is a graph of a photonic sensor system-on-a chip transdermal blood glucose sensing performance using the data processing method of an embodiment of the invention, with a sedated piglet;

FIG. 11 is a graph of a photonic sensor system-on-a chip transdermal blood lactate sensing performance using the data processing method of an embodiment of the invention, with a sedated piglet; and

FIG. 12 is a graph of a photonic sensor system-on-a chip transdermal blood ethanol sensing performance using the data processing method of an embodiment of the invention, with a sedated piglet.

FIG. 13 is a graph that illustrates the effect of Kubelka-Munk preprocessing on the transdermal tissue spectrum while analyzing ethanol.

FIG. 14a illustrates decomposition of an observed transdermal signal using the Beet-Lambert model, without pre-processing the observed signal using Kubelka-Munk correction.

FIG. 14b illustrates decomposition of the same observed transdermal signal, using the same Beet-Lambert model, but after pre-processing the observed signal using Kubelka-Munk correction.

### DETAILED DESCRIPTION

Optical remote sensing is a developed technique for a broad range of applications. Sensing can be performed as a form of ranging—i.e., measuring distance by means of time-of-flight or frequency modulated continuous wave (FMCW) technique, or sensing can be performed to remotely detect, identify, and quantify the presence or absence of one or more molecules within an object by spectroscopic sensing.

The term spectroscopic sensing, as used herein, refers to deployment of a hybrid III-V/IV semiconductor photonic system-on-a-chip (P-SoC), which emits wavelength tunable laser radiation and is in communication with a remote target object. The wavelength change and absolute value are monitored and accounted within every sweep, such that the SoC is autocalibrated in terms of absolute wavelength and wavelength shift and power spectrum.

The light impinges the object and penetrates to a certain depth, defined by the optical length, which depends on the individual specifity of the object such as scattering matrix, content, etc. For example, using tunable laser radiation in the 1900-2500 nm spectral region to perform a transdermal sensing experiment with a living object, the light penetrates up to about a 1 mm below the skin surface, where it is scattered and is partially absorbed by the tissue, blood, and interstitial fluid. Such absorption is molecule-specific and each constituent molecule modifies the light spectrum with a unique spectral absorption signature. After interaction with the object, the transmitted, scattered or reflected light is collected and detected with a photodetector.

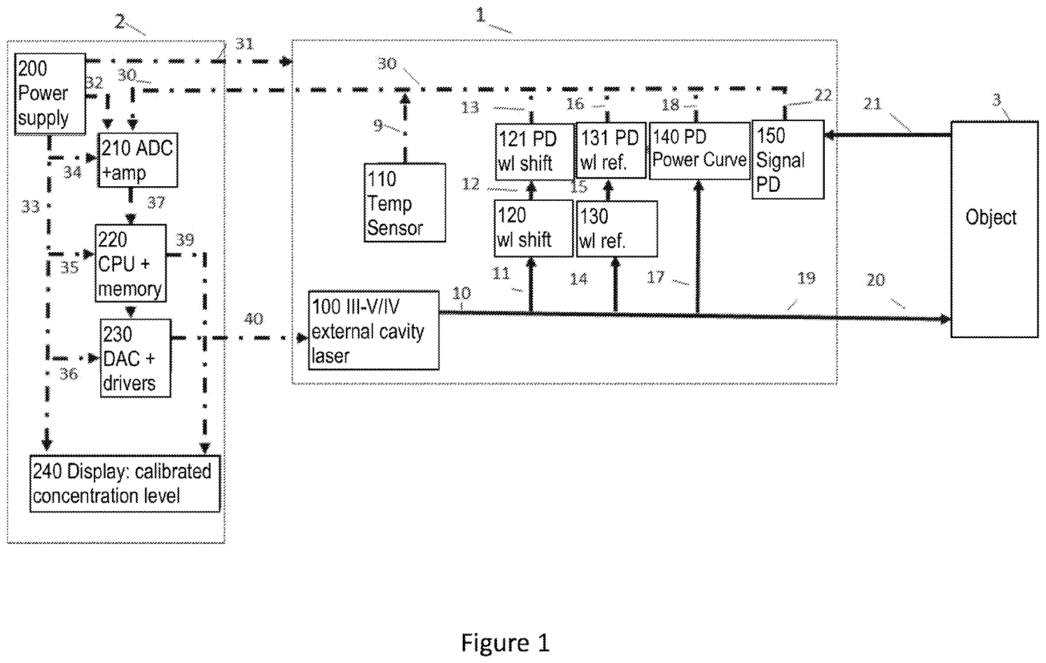

A schematic block diagram describing an embodiment of the invention is shown in FIG. 1. Here the photonic system-on-a-chip includes a hybrid III-V/IV semiconductor chip 1 and control and signal processing electronics 2, which form the hardware part of the photonic sensor chip. The photonic sensor on a chip is in optical communication with object 3, which can be a living body, isolated substance, etc. Within the configuration, the photonic system on a chip is remote from the object.

In the illustrated embodiment, the hybrid III-V/IV semiconductor chip 1 includes a hybrid III-V/IV external cavity laser 100, which emits swept-wavelength laser radiation via an optical path 10. A portion of the beam is split via path 11, and fed into the wavelength shift tracker 120 via path 11, absolute wavelength reference 130 via optical path 14, laser power curve monitoring block 140 via optical path 17 and output section via optical path 19. Chip 1 may also include a temperature sensor 110, for sensing the temperature of the chip, which in turn can be used for absolute wavelength reference calibration.

Wavelength shift tracker 120 can be any type of non-balanced interferometer such as Mach-Zender, Michelson, Fabry-Perot, etc. A non-balanced interferometer provides a beat signal at the output of the 120 via the optical path 12, and the photodetector block 121 register an oscillating signal, where the oscillation period depends on the optical path difference within the interferometer and wavelength. The optical path difference is defined by the design and is a known parameter. The wavelength shift value can thus be extracted if the absolute value of the wavelength at any given moment is known. This is provided by the absolute wavelength reference block 130, coupled to monitoring photodetector 131 via optical path 15. The absolute wavelength reference can be a distributed Bragg grating (DBR), micro-ring resonator (MRR), distributed feedback grating (DFB) or any other optical cavity structure with unambiguous characteristic transmission or reflectance feature within the spectral region covered by the hybrid laser 100 sweep. In such a way, the photodetector blocks 121 and 131 cooperatively provide information about the absolute wavelength value and wavelength shift value at any given moment of time within the sweep.

Tracking wavelength shift and absolute wavelength value is often necessary in order to decouple the system effects from the object related effects. For example, the emission wavelength might change in a non-linear manner on the system side, and thus without precise knowledge of absolute wavelength shift and value information, it can be difficult to perform signal conversion from time-domain to wavelength (or frequency domain). Another aspect is that the collected spectra will change due to changes on the object side—such as water displacement due to temperature, or changes in other strong baseline contributors. Without knowing the system output at all times, it is impossible to decouple whether the collected spectrum from the object is shifted due to changes at the output of the system or influenced by the changes within the object. Therefore, wavelength shift and absolute wavelength information tracking within every sweep allows one to decouple the system specific modulation on the collected spectra from the object specific modulation, the latter being the useful signal.

In practical cases, the target molecules such as glucose, lactate, ethanol etc. have concentrations which are very small compared to the main baseline contributors, which for the case of transdermal sensing are main proteins (collagen, albumin, keratin) and water. These main contributors provide signal which is 10 000 or more times stronger compared to the target molecules, and thus a small change in water displacement due to temperature effect can lead to the baseline change that if unnoticed can smear out any useful signal that can be attributed to glucose. Therefore, the ability to track the wavelength shift and absolute value within every sweep allows access to tracking the baseline change within every sweep.

The wavelength shift may be monitored as the beat signal during the sweep, whereas the absolute value is measured once per sweep, and the information from both the wavelength shift and absolute wavelength is used to calibrate the recorded information immediately afterwards the sweep is complete. The accuracy for the determination of the wavelength shift depends on the system design such the optical path difference within the wavelength shift tracker which in turn provide a beat signal. In a practical case scenario, this depend on the finesse of the absorption feature of the target molecular specie within the object. In case where the object is a biological substance and the molecules represent liquid phase, which are characterized by very broad spectroscopic signatures, the wavelength shift tracker can have the accuracy of 0.1 nm to few nm., 3-5 nm being a typical value.

In case of gas sensing, where the absorption line width of interest can be in the range of 100 MHz, the, wavelength shift tracking needs to be designed to possess a better resolution and the absolute wavelength reference needs to be designed to provide the absolute wavelength with a high enough resolution. In practical cases, this can be achieved with a very good accuracy. For instance, typical group-IV semiconductor fabrication technologies rely on node sizes as low as 160 nm or even down to 7 nm, which is three orders of magnitude compared to a typical emission wavelength. The time duration for one sweep is defined by the system architecture and last from minutes, when the tuning mechanism is performed by a mechanical motion of the tuning element, to few micro seconds, if the tuning is electronic. In a practical case for a hybrid III-V/IV sensor chip, the sweep rate can be from few tens of Hz up to MHz range, depending on actual practical system design and the application requirements.

Depending on the sensor design, and the requirement for the spectral bandwidth coverage, a single sweep can contain from several 10s to several 100s of discrete wavelengths. A typical practical case for transdermal glucose sensing requires around 100 or more discrete wavelengths to perform accurate prediction. Based on the existing state-of-the art widely tunable (swept-wavelength) laser concepts, the sweep can be almost continuous when the Vernier-filter is operated in combination with phase control. In some embodiments, the absolute value of the wavelength of emission is tuned within a specified range, e.g., 1000 to 3000 nm, 1900 nm to 2500 nm, etc. Thus, the tuned value of the wavelength of emission at a particular time may be 1898 nm, 1905 nm, etc. The corresponding wavelength shift can be 1 nm, 2 nm, 10 nm, etc.

The EMR received from the medium of interest is converted from the optical domain into electrical signals within the photodetectors 121 and 131, and the electrical signal from the photodetector is routed via electrical paths 13 and 16 to the electrical path 30 which connects to the drive and control electronics block 2, and analog-digital-converter (ADC) and amplifier block 210 therein. Here, the analog signal from the photonic chip is amplified and digitized. Digitized signal is fed into CPU 220, which performs signal filtering, averaging and other processing The CPU 220 contains a memory block with a calibration model. This calibration model is applied to the collected data to retrieve a calibrated concentration level value, which is then fed to an output port, e.g., the display 240 via electrical route 39. Another function of the CPU 220 is to provide control signals to the driver and digital-to-analog converter (DAC) block 230 via path 38, which in turn provides control and drive signals to the SoC via path 40. The entire sensor system is powered by the power supply 200 via electrical bus 31, 32, 33, 34, 35, 36.

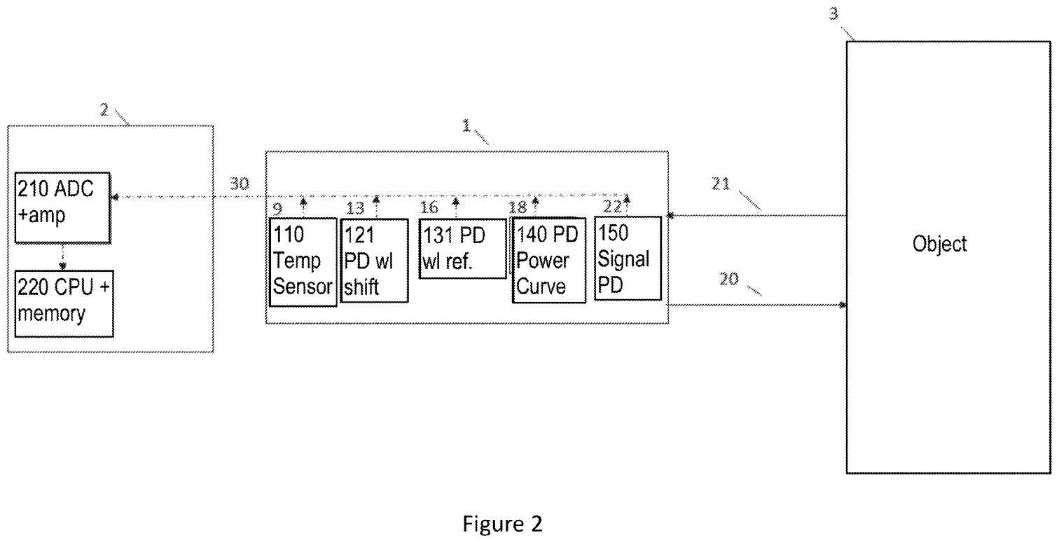

A simplified version of the sensor system of FIG. 1 is provided in FIG. 2, with several internal blocks such as analog-to-digital converter (ADC) block 210, wavelength shift tracker photodetector block 121, absolute wavelength reference photodetector 131, laser power curve photodetector 140, signal photodetector 150 and CPU 220 highlighted separately for clarity.

When deployed in the field, the photonic sensor on a chip 1 sends a wavelength tunable signal to a remote object 3 via optical path 20. The intensity I of the signal can be represented as an arbitrary function of frequency ω (or wavelength) and time t:

I=ƒ(ω,t) (1)

The light interacts with the object 3, and undergoes numerous scattering and absorption events within the object. A portion of scattered and diffusely reflected light is collected with signal photodetector 150 via the optical path 21. This light signal intensity can be represented by a frequency and time function I′:

I′=ƒ′(ω,t) (2)

This signal is modulated due to the interaction with the object and carries object-specific information such as concentration level of constituent elements. The latter can be evaluated as absorbance A, which can be represented as a linear superposition of individual absorbances Aᵢ:

$$A = - ln ( \frac{{I}^{′}}{I} ) = {\sum }_{i} {A}_{i} = {\sum }_{i} {ε ( ω )}_{i} {c}_{i} l = {ε ( ω )}_{1} {c}_{1} l + {ε ( ω )}_{2} {c}_{2} l + {ε ( ω )}_{3} {c}_{3} l + … \qquad ( 3 )$$

Here, ε(ω)ᵢ is frequency dependent individual molar absorptivity of a constituent i, cᵢ—is individual molar concentration of the constituent i and l—is the effective optical length within the object.

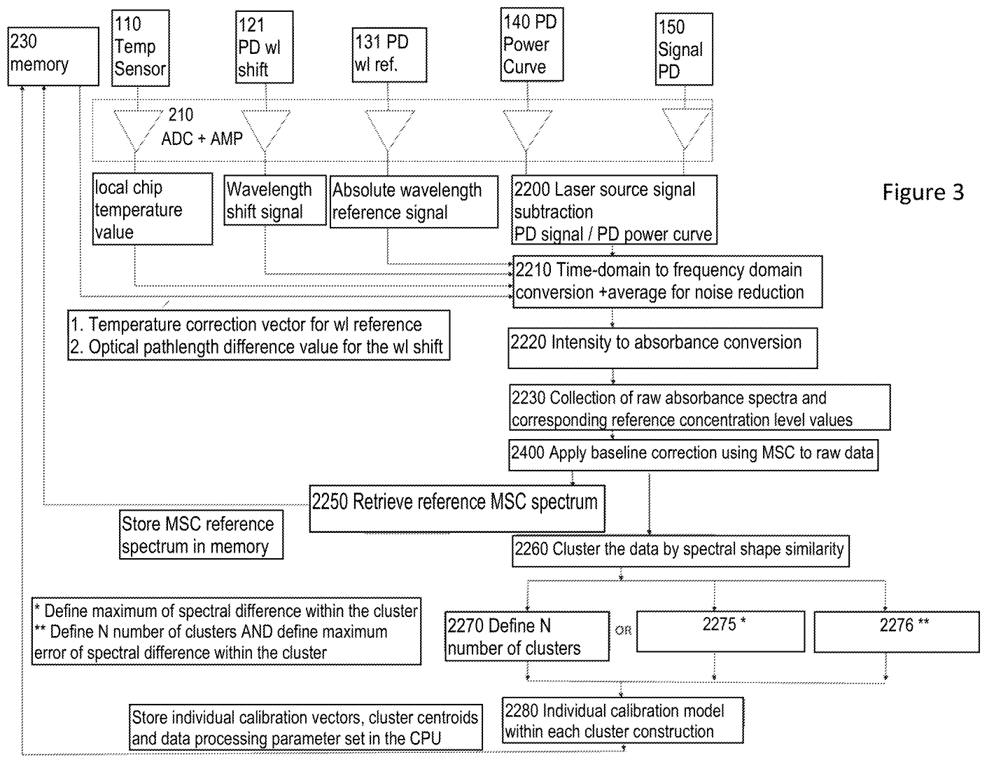

In a practical case, where the object is a living body, the individual absorbance contributions can be represented as contributions by different constituent elements such as for example: 1—keratin, 2—glucose, 3—lactate, 4—urea, 5—collagen, etc. This provides a path of elemental decomposition of complex matrix and thus offers a possibility for sensing. The procedure for collecting and processing data and deriving calibrated concentration values is shown in the form of a block diagram in FIGS. 3, 6 and 9.

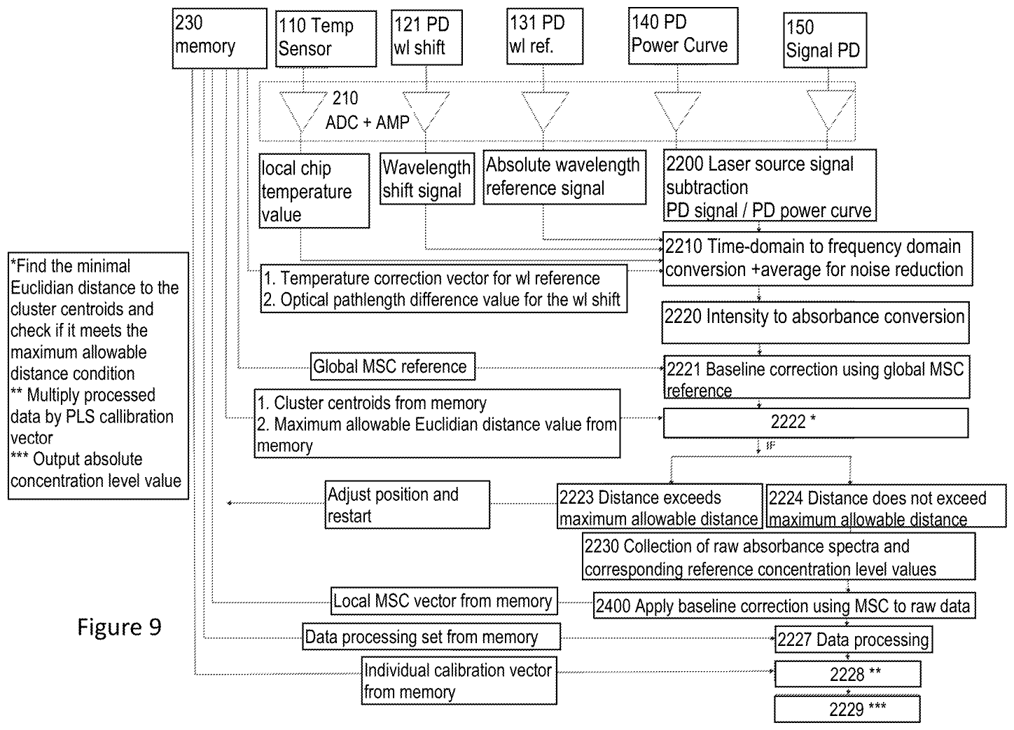

The basic operation method for performing sensing includes first using the calibration algorithm in combination with the hardware to create a calibration model and store it in the memory of the CPU. This model can be considered universal and be deployed with every sensor in the field without needing to modify it during use. The next step is to then use the sensing algorithm in accordance with FIG. 9, in combination with the hardware and the calibration model stored within the system memory or CPU.

According to an embodiment of the invention, when deployed in the sensing configuration, the photonic system on a chip provides several output channels that contain information about the state of the photonic chip, such as wavelength shift value via photodetector 121, absolute wavelength reference value via photodetector 131, laser intensity curve via laser power curve monitoring block 140, and/or the reflected signal containing object-specific information via signal photodetector 150. These electrical signals are routed to the control and signal processing electronics block 2. Here, the signals are fed into the analog-digital-converter and amplifier block 210.

### System Calibration for Analyte Measurement

The algorithm for processing the acquired analog signals received from the photonic SoC 1 starts by first amplifying and digitizing the received signal in the ADC and amplifier block 210. At this phase the signals are still processed as time domain signals. These amplified and digitized signals are then fed to the central processing unit (CPU) 220, where the object-specific signal 22 is processed and converted from time-domain into the frequency-domain using the information of wavelength shift received via electrical path 13, and absolute wavelength calibration received via electrical path 16, and is normalized with regard to the laser power curve received via electrical path 18. This procedure allows first having the signals in the frequency domain and also addresses the system-related nonlinearities, to further process the signal that primarily carries object-specific data, indicated as step 2210 in FIG. 3.

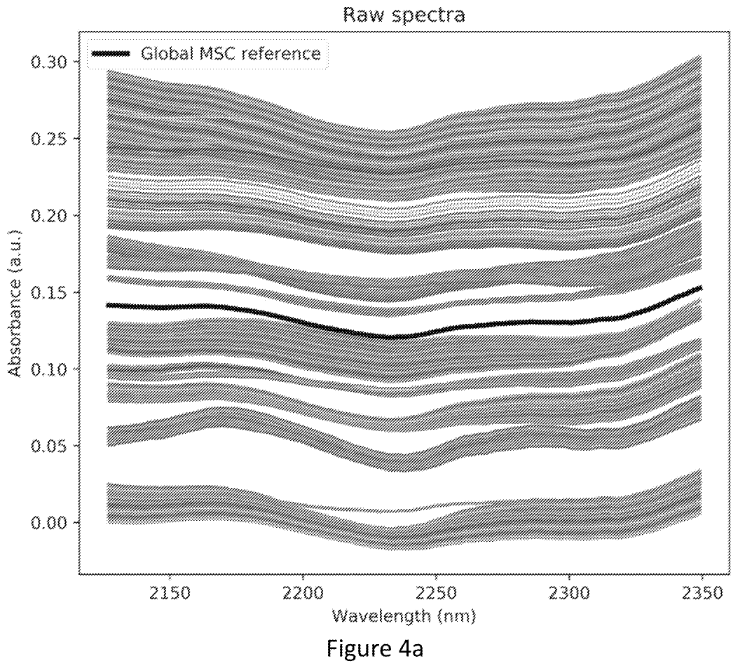

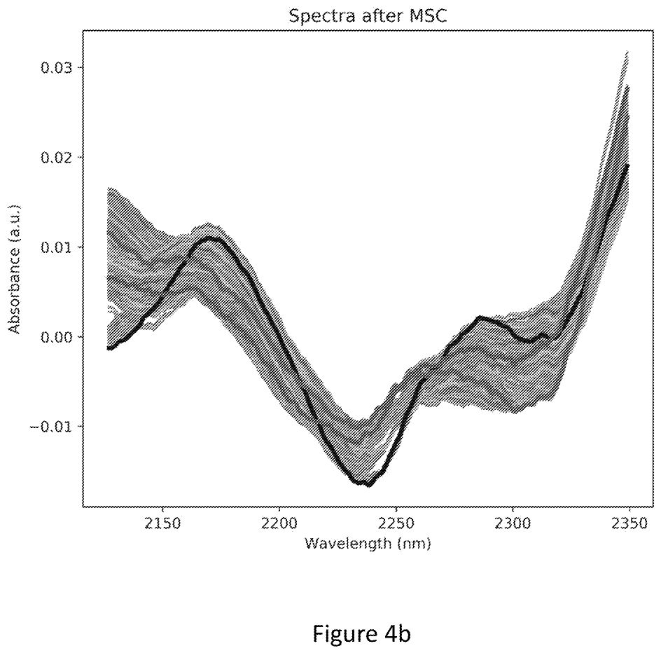

Multiple spectra are collected, averaged, and filtered to reduce noise. For example, in FIG. 4A each individual curve represents an averaged spectrum. Afterwards, the corrected intensity is converted to absorbance as per equation (3) within step 2220 and a large number of raw absorbance spectra is accumulated as indicated per step 2230. Raw spectra typically have a large variety of spectral shapes due to different tissue physiology (e.g., from different tissue samples having different scattering particle size, etc.) and intensity due to optical path length differences and/or particle size differences. To correct for the scattering effects, multiplicative scattering Correction (MSC) is applied to the raw absorbance data within 2240 and the global MSC reference file (or mean spectrum), is extracted within step 2250 (indicated as a bold line roughly in the center of the graph of FIG. 4a) and then stored in the system memory. This global MSC spectrum is later used to assign the raw data into the right cluster based on exactly the same baseline correction procedure. Then, all the baseline corrected data (after MSC for example) within step 2240 (see FIG. 4b) is next grouped into clusters based on the spectral shape similarity in step 2260. Other types of scattering correction techniques, such as standard normal variate (SNV) correction, Kubelka-Munk correction, Saunderson correction or mean centering and normalization correction, may be applied as an alternative to multiplicative scattering correction.

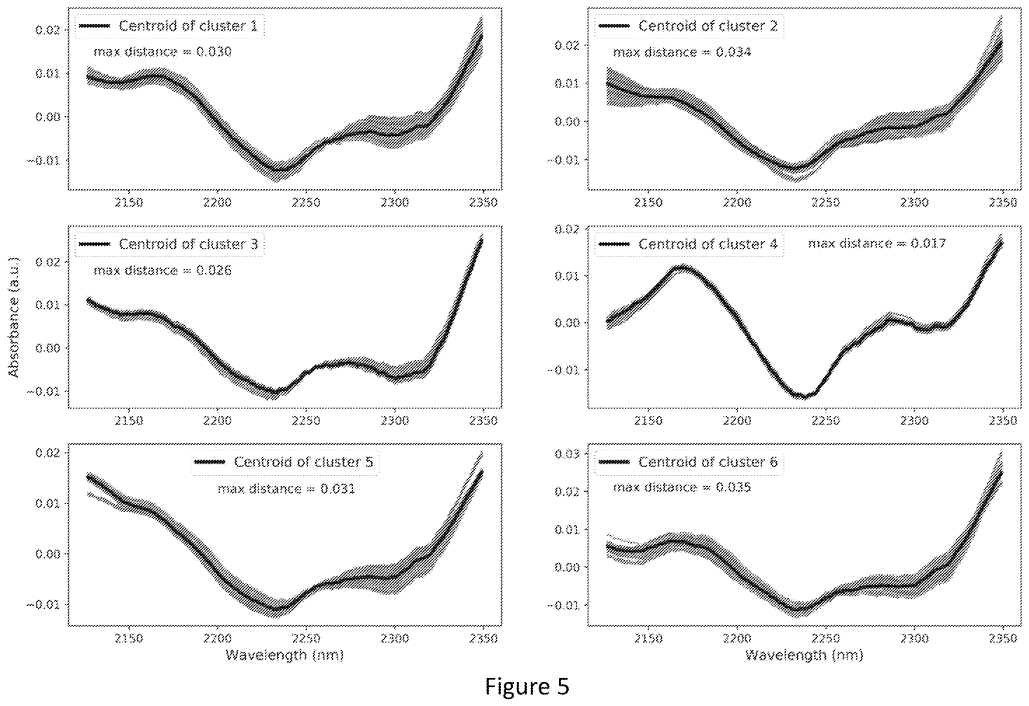

Referring to FIG. 5, baseline corrected spectra from FIG. 4b are clustered into six different clusters (N=6 by definition) using k-means algorithm. A maximum distance to the centroid within each cluster is indicated within the graph.

As illustrated, the global MSC corrected data is only used for assigning raw spectra to each cluster. Thus, the assigned cluster contains raw or unprocessed data. Clustering can be performed in a number of ways. Two possible paths are shown in FIG. 3. In the first path, i.e., step 2270, the spectra are grouped to a fixed and defined number of clusters N based on spectral shape similarities. The downside of this approach is that the error, or the distance from spectra to the assigned cluster centroids σ^{k}, where σ is the array of cluster centroids, and k is the cluster number, may vary greatly among different clusters (see FIG. 5). In a potentially better path, shown as step 2275, clustering is performed by defining the maximum distance from any spectra to the cluster centroid, resulting in an arbitrary number of clusters, which in practical case can be large. Thus, an intermediate route may be used, indicated as 2276, where both the number of clusters N and the maximum allowable distance to cluster centroids is defined. In such case, spectra that do meet the distance to centroid criteria within the defined number of clusters are considered as outliers and are not used in the data analysis. While the predefined number of clusters can be arbitrarily large, in practical cases it may be 10-50 depending on the analyte and sensing geometry. The set of cluster centroids is stored in the CPU memory, which will later be used for the sensing function to assign the GS corrected spectra.

Once the clustering is complete, an individual calibration model within each cluster is created at step 2280. An individual calibration model assigns a calibrated concentration level value to every spectrum within each cluster as measured by the gold standard as indicated. This set of calibration models is then stored in the CPU memory next to the MSC reference vector in step 2300.

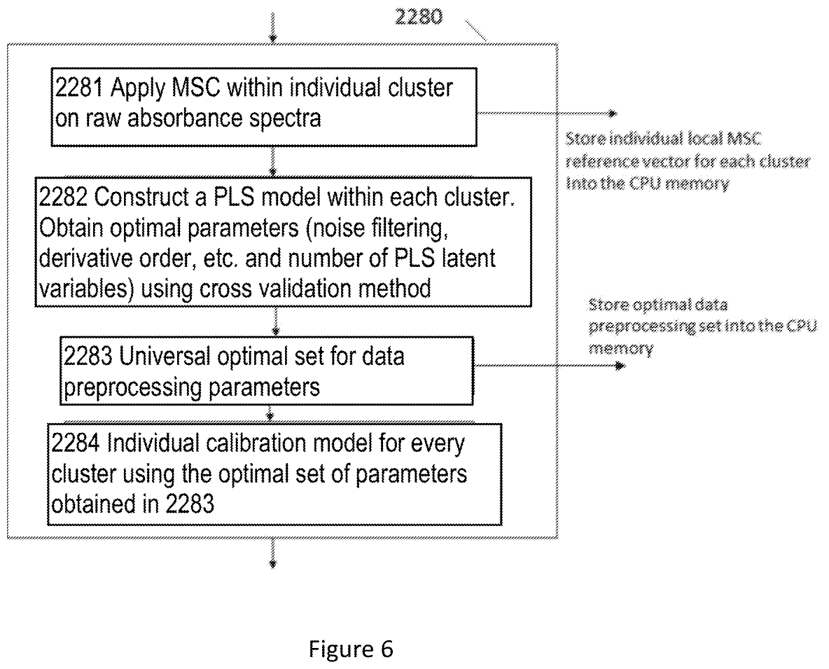

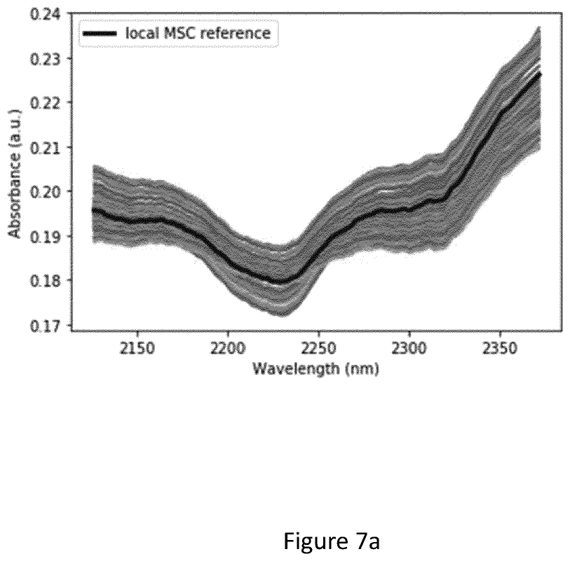

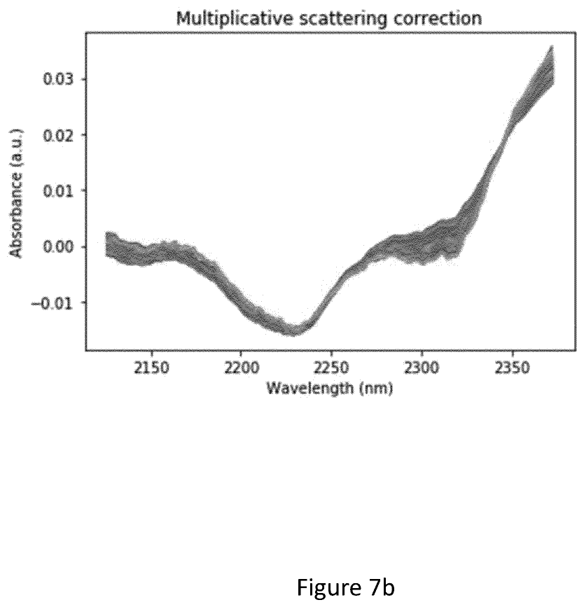

The algorithm for constructing an individual calibration model 2280 is depicted in FIG. 6. Referring also to FIGS. 7a and 7b, according to an embodiment of the invention, the step 2280 of construction of an individual calibration model within each cluster starts by applying scattering correction (MSC for instance) to the raw spectra within with the cluster. This yields an individual, local MSC reference for each cluster, which is stored in the CPU memory (see FIG. 7a bold line). This local reference is used to process the acquired raw data in sensing mode. Other types of scattering correction techniques, such as standard normal variate (SNV) correction, Kubelka-Munk correction, Saunderson correction or mean centering and normalization correction, may be applied as an alternative to multiplicative scattering correction.

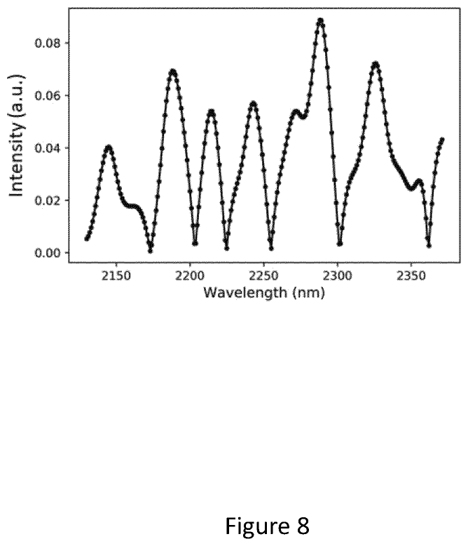

The local reference from 2281 is then used to construct a partial-least-square (PLS) model within each cluster and obtain optimal model parameters such as noise filtering parameters, derivative order, number of PLS latent vectors using a cross-validation method within step 2282. The task yields an optimal set of data preprocessing parameters 2283 that are then applied to every cluster containing raw spectra to construct individual calibration model 2284. In other words, within each cluster, the raw spectra are modified using the local scattering correction reference. This ensures that all data is treated in the same manner, with the same set of parameters. A calibration model then assigns a calibrated concentration level(s) of the analyte(s) of interest that is/are measured by a selected reference technique (also called gold standard) to each locally corrected spectrum. The calibration model maps the absorbance represented by a spectrum at a particular wavelength to an analyte concentration level. Referring to FIG. 8, the obtained individual calibration vectors are then stored in the CPU memory. The calibration vector is an output of multivariate regression calibration. After model training using all spectra within a cluster, it determines weights for locally corrected and preprocessed absorbance spectrum values at each wavelength. In prediction, every i-th wavelength value of preprocessed absorbance is multiplied by corresponding weight and then by summing across all the wavelengths we get a predicted concentration as:

c=w₁A₁+w₂A₂+ . . . +wₙAₙ, where n is the number of wavelengths in the spectrum. In some cases, when the sample is associated with a relatively simple scattering matrix, and when the sample includes fewer constituents, a reasonable concentration prediction can be obtained simply by preprocessing the spectral data obtained from the sample to correct for the nonlinear effects of scattering using Kubelka-Munk correction, MSC, Saunderson correction, or a combination thereof, and then by removing the baseline to obtain the spectrum of a constituent of interest. For greater accuracy and, especially for more complex samples such as biological tissue, scattering correction (or linearizing transformation) may be used in combination with multivariate linear regression such as PLS or similar.

In general during calibration, EMR is directed to a sample (also called medium), where the EMR is swept through a range of wavelengths. In response, EMR is received from the sample, where the received EMR is diffusively reflected by or is transmitted through the sample. The received EMR, having components at different wavelengths, is converted into a raw absorbance spectrum (also called a raw spectrum). This process may be repeated several times to obtain a number of raw spectra, which are then averaged to obtain an averaged raw spectrum. In the discussion below, we omit the term “averaged” for simplicity. These raw spectra may be denoted Xᵢ^{raw}, where the index i denotes respective, averaged raw samples and can range from 1 to M, where M can be any number such as 50; 100; 2000; 10,000, or more. The above-described process is repeated at different times, where the analyte concentration in the sample may be different at different times, using different regions of the samples or different samples, where the analyte concentration may be different in different regions of the same sample or in different samples.

Scattering correction (MSC, Kubelka-Munk correction, Saunderson correction, etc.) is then applied to the raw spectra Xᵢ^{raw}, to obtain a global reference denoted X_{ref}^{G} and, globally corrected spectra Xᵢ^{GC}. Global reference X_{ref}^{G} is stored in the memory. Clustering is then performed using the globally corrected spectra Xᵢ^{GC} to identify N clusters. The number N (e.g., 4, 5, 6, 10, etc.) may either be specified for the clustering operation or, alternatively, the clustering itself may determine the best N. For each Xᵢ^{GC}, a corresponding cluster Cₖ, k∈[1,N], is identified and, thereafter, the corresponding raw spectrum Xᵢ^{raw} is designated to the same cluster. After clustering, the optimal number of clusters, cluster centroids and maximum allowable distance to cluster centroids are stored into the memory to be used for the sensing function.

Once all the raw spectra are designated to their respective clusters, within each cluster the above-described process is repeated. Specifically, scattering correction is applied to the raw spectra Xᵢ^{raw} within a particular cluster k, to obtain a local reference denoted X_{ref}^{L}^{ₖ}, which is stored in the memory. By locally applying scattering correction to the raw spectra Xᵢ^{raw}^{ₖ} within the cluster k, locally corrected spectra Xᵢ^{LC}^{ₖ} for the cluster k are generated. This process is repeated for all clusters, to obtain the respective local references X_{ref}^{L}^{ₖ} and locally corrected spectra Xᵢ^{LC}^{ₖ} for each k∈[1,N].

Recall, the different raw spectra Xᵢ^{raw} may correspond to different levels of analyte concentration. These concentrations levels, denoted [symbol] ᵢ, are obtained from the samples using a selected gold-standard technique. Finally, a calibration vector V^{k} is generated for each cluster k via multivariate linear regression calibration. The calibration vector V^{k}, local reference X_{ref}^{L}^{ₖ}, and data preprocessing set used to generate the calibration vector may be stored in a memory module in the SoC for each cluster. Data preprocessing set defines whether the calibration vector was obtained using absorbance, absorbance treated with n^{th} order of derivative, order of filtering, Kubelka-Munk correction, Saunderson correction, Multiplicative Scattering Correction etc. This is necessary to ensure that all raw data is treated in exactly the same way, when the sensor is deployed for sensing. The global reference X_{ref}^{G} may also be stored in the memory module of the SoC.

One example process for obtaining an optimal data preprocessing set is as follows:
1. Within a cluster, apply signal smoothing (noise filtering) with iteratively selected filter and its degree (e.g., Savitzky-Golay, Fourier transform filter, percentile, moving average) on locally corrected spectra. Additionally, 1^{st} or 2^{nd} order derivative baseline removal may also be applied.
2. Locally corrected and preprocessed spectra and corresponding concentrations are randomly divided into the training and test sets.
3. Multivariate regression calibration algorithm is applied on training set and after model is trained, prediction of concentration is performed using test set and prediction accuracy is evaluated.
4. Steps 2 and 3 are repeated a number of times (e.g., n iterations) in a process called cross-validation to get an average prediction accuracy for current data preprocessing set.

Steps 1-4 may be repeated with different sets of parameters selected in step 1. An optimal set of parameters is the set that resulted in best average prediction accuracy.

A multivariate regression algorithm models a relation between predictor and response variables. Thus, a calibration spectral matrix χ∈ [symbol] ^{d} may be considered as predictor, where d is a number of wavelengths, and analytes concentration vector [symbol] ∈ [symbol] is considered as a response. Each i^{th} row of spectral matrix corresponds to locally corrected and preprocessed spectrum (e.g., Savitzky-Golay filter and second derivative applied on locally corrected absorbance spectrum) and each i^{th} row of response vector corresponds to analyte concentration measured with the gold standard. Once the relation between predictor and response is determined, unknown value of analyte concentration can be predicted based on the new locally corrected and preprocessed spectrum. Multivariate regression may include partial least squares regression and its modifications, multiple linear regression, support vector regression, artificial neural networks, and/or principal components regression.

### Sensing or Analyte Measurement

Referring to FIG. 9, individual calibration vectors, global MSC reference, and local MSC vectors stored within the memory allow performing sensing function of the hybrid photonic SoC by means of the sensing algorithm. In particular, once deployed in the field, the hybrid III-V/IV photonic SoC, collects diffuse reflectance signals, which are then, together with the absolute wavelength reference, wavelength shift value and laser power curve signals, are amplified and digitized within the ADC+amp section 210. Then the time-domain signals are converted into frequency domain, averaged and calibrated in terms of absolute wavelength, wavelength shift, chip temperature and laser power curve within step 2210. Next, the reflected intensity is converted to absorbance in step 2220.

Next, the collected absorbance spectra undergo baseline correction, using global scattering correction GSC reference taken from the CPU memory in step 2221 in order to initiate the clustering procedure. For clustering the collected spectra, cluster centroids and maximum allowable distance to the cluster centroids are provided from the CPU memory, and the data is classified accordingly in step 2223. If the distance to the provided cluster centroids exceeds a maximum allowable distance, the CPU initiates an error message to indicate to the user to adjust the sensor position and restart the data collection until the error is not larger than the maximum allowable, in step 2224. If the collected data, after baseline correction, has the distance to cluster centroids within the allowable range in step 2225, the collected corresponding raw spectrum is assigned to a cluster with minimal distance to the centroid in step 2226.

Next, in step 2227, the raw spectrum within the newly assigned cluster undergoes baseline correction using a local scattering correction reference from the CPU memory and the data is preprocessed using the data processing set from the CPU memory in step 2228 in order to be eligible for data prediction step 2229, where it is multiplied with the individual calibration vector V^{k} from the CPU memory obtained by a multivariate regression calibration. Multiplying row vector of spectrum with column vector of regression weights, we obtain a single value for analyte concentration. Each different analyte will have different calibration vector and thus weights—i.e., different wavelength specificity for a particular analyte. For instance, 2100 nm can be relevant both for lactate and glucose, however the weights will be different. Analyte's concentration is c=w₁*A₁+w₂*A₂+ . . . +wₙ*Aₙ. Here, wₙ is the calibration weight at nₜₕ wavelength and Aₙ is locally corrected and pre-processed absorbance at nₜₕ wavelength. The output is then a calibrated concentration level of the analyte of interest.

In general the sensing process starts in a similar way as the calibration process. Specifically, EMR is directed to a sample (also called medium) from where analyte concentration is to be determined. The EMR is swept through a range of wavelengths. In response, EMR is received from the sample, where the received EMR is diffusively reflected by or is transmitted through the sample. The received EMR, having components at different wavelengths, is converted into a raw absorbance spectrum (also called a raw spectrum). This process may be repeated several times to obtain a number of raw spectra, which are then averaged to obtain an averaged raw spectrum denoted Y_{raw}. Here again in the discussion below, we omit the term “averaged” for simplicity.

Scattering correction is then applied to the raw spectrum Y^{raw} using the global reference denoted X_{ref}^{G} (generated during the calibration process), to obtain a globally corrected spectrum Y^{GC}. Clustering is then performed using the cluster centroid value σ^{k} and the distance to the centroid value from the memory. That cluster may be denoted Cₖ, where k∈[1,N], and where the number N was either specified for the clustering operation or, alternatively, was determined while performing clustering as part of the calibration process. The corresponding raw spectrum Y^{raw} is then designated to the same cluster Cₖ.

Thereafter, scattering correction is applied again to the raw spectrum Y^{raw} within the selected cluster Cₖ, using the corresponding local reference denoted X_{ref}^{L}^{_{c}}. By locally applying scattering correction and the data pre-processing parameters set to the raw spectrum Y^{raw} within the cluster Cₖ, a locally corrected and pre-processed spectrum Y^{LC} is generated. Using the absorbance values of the spectrum Y^{LC} and the calibration vector V^{k} for the selected cluster Cₖ, a concentration level for the analyte of interest is estimated. This overall process may be repeated a number of times, to obtain several estimates of the analyte concentration, to provide an averaged estimated analyte concentration.

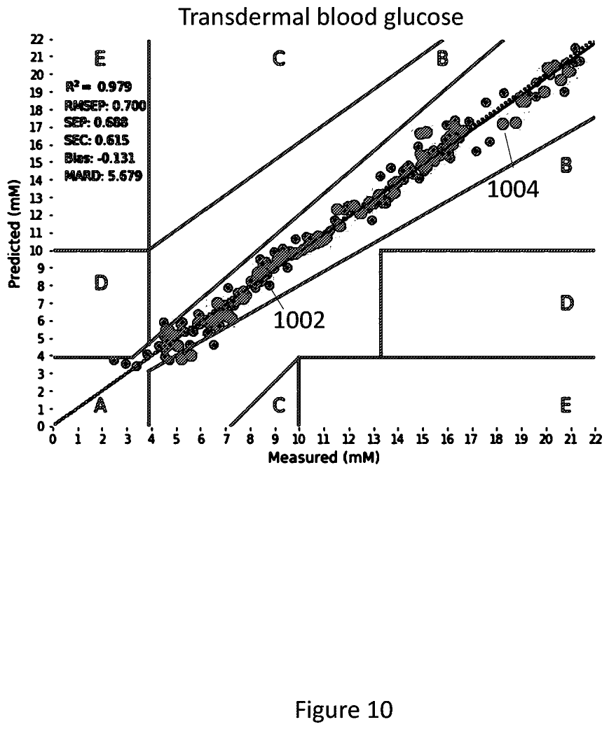

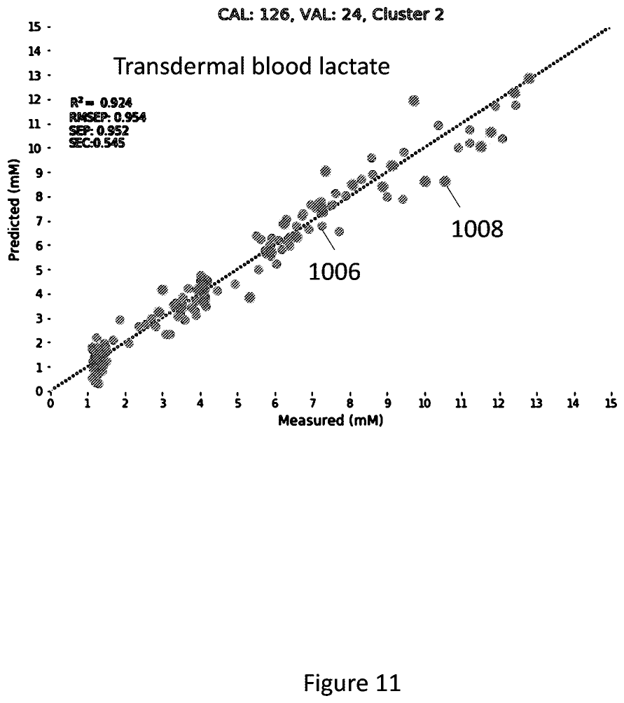

An example of a transdermal sensor performance with a piglet for three different analytes—i.e., blood glucose, blood lactate and blood ethanol, in accordance with an embodiment of the invention, is provided in FIGS. 10-12. Here, for all experiments, an approximately 40 kg female pig was sedated for a duration of 8 hours, and a buffered analyte solution glucose solution was injected into a vein to raise the blood analyte level of the pig. In case of glucose, FIG. 10, blood glucose level was raised by injecting the buffered glucose solution and insulin was administered to lower the blood glucose level. In case of lactate, FIG. 11, the blood lactate level was raised by injection into a vein, and lowered by means of terminating the buffered lactate administration, allowing the pig to clear the lactate level naturally. For the case of ethanol, again the blood ethanol level was raised by injecting a buffered solution into a vein, and lowered by terminating and allowing the body to clear ethanol naturally. In all cases, the III-V/IV sensor was in contact with the skin of the pig at the belly of the sedated pig. The sensor was sampling the pig at a frequency of 40 Hz (40 sweeps/s or 40 spectra/s). Blood sample was drawn from the artery of the pig every 6 minutes, and analyzed with a clinical analyzer used as a gold standard. In the described embodiment we used two Abaxis Piccolo Xpress analyzers for blood glucose calibration, EKF Biosen C_line analyzer for lactate calibration and Agilent 8860 gas chromatographer for blood ethanol calibration as clinical gold standard. Collected spectra were then assigned to a calibrated glucose concentration level value as measured with the gold standard and data was processed according to the procedure described in the embodiment of the invention.

In FIG. 10, the data points 1002 represent the data points that were used to form the calibration model, and the red data points 1004 represent multiple prediction using that model for the particular pig under study. In this case the model and validation uses the data acquired from the same pig. The blood glucose level of the pig was ramped up and down during the duration of the day, and calibrated data was measured every 6 minutes using the gold standard. Optical spectra that are collected in between the two calibrated points were interpolated and assigned an absolute glucose concentration value.

The representative results, demonstrate excellent sensor's performance in a wide dynamic glucose concentration level range from 75 mg/dl (4.16 mmol/l) until 400 mg/dl (22.2 mmol/l), the determination coefficient of 97.2%, root mean square error of prediction (RMSEP) of 14.7 mg/dl (or 0.8 mmol/l) and mean absolute relative difference of 6.7% in the entire range.

In FIG. 11, the green data points 1006, represent the data points that were used to form a calibration model, and the red data points 1008 represent multiple prediction using that model for the particular pig under study. In this case, the model and the validation uses the data from the same pig. The representative results demonstrate transdermal blood lactate sensing in the 1 mmol/l-15 mmol/l concentration level range with a determination coefficient of 92.4%, and RMSEP of 0.954 mmol/l.

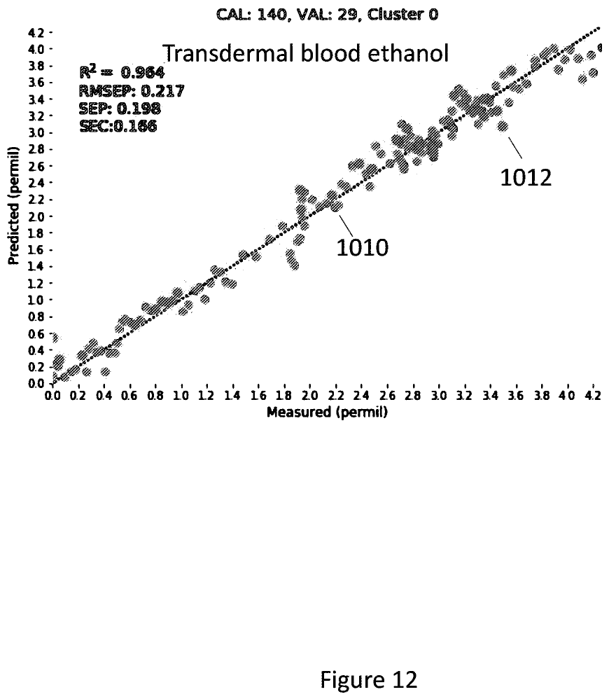

In FIG. 12, the green data points 1010, represent the data points that were used to form a calibration model, and the red data points 1012 represent multiple prediction using that model for the particular pig under study. In this case, the model and the validation uses the data from the same pig. The representative results demonstrate transdermal blood ethanol sensing in the 0.2‰-4.2‰ concentration level range with a determination coefficient of 96.4%, and RMSEP of 0.217‰-

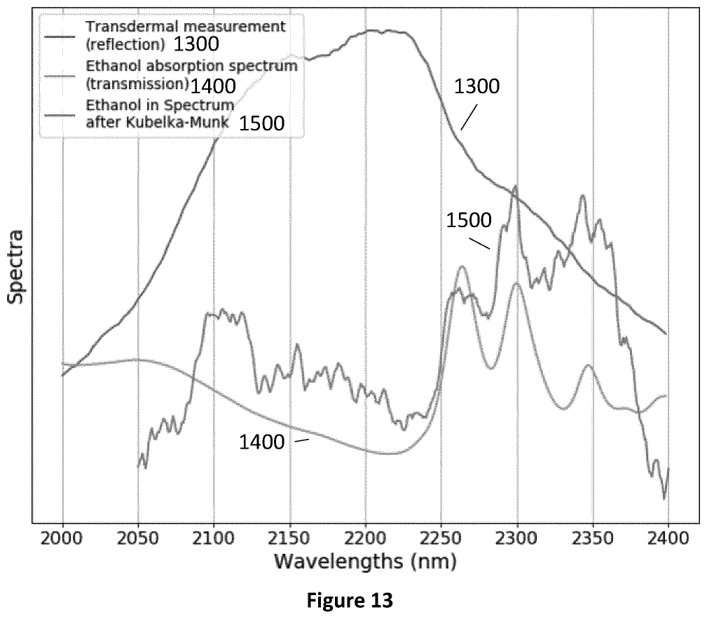

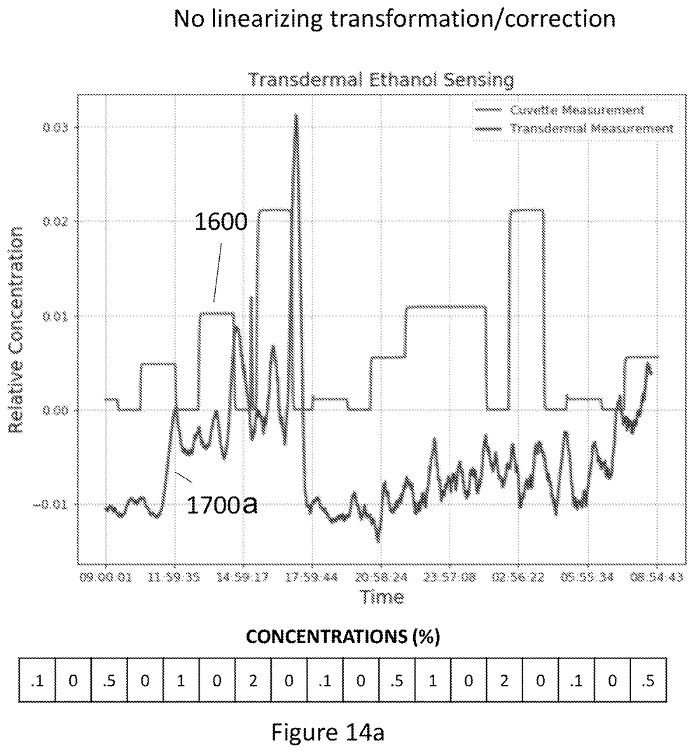

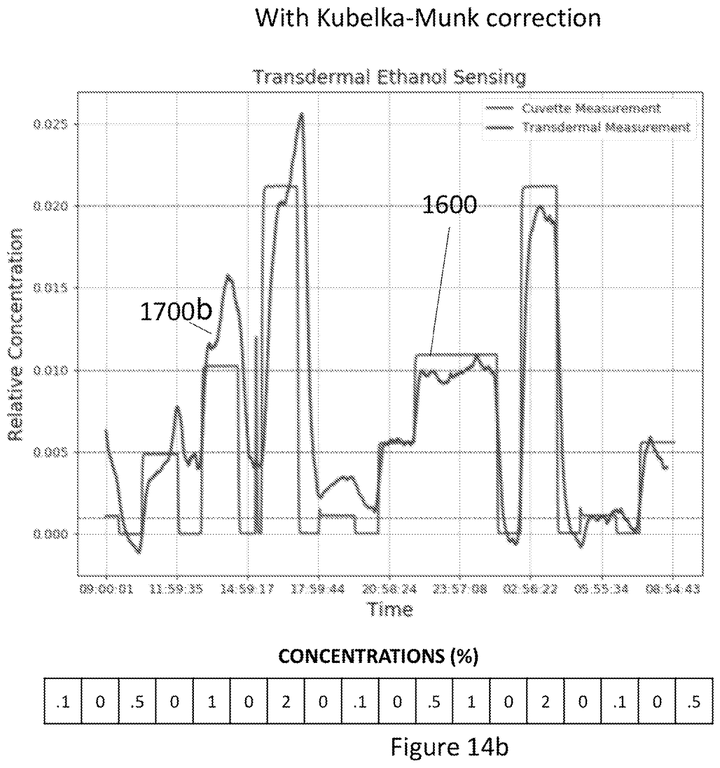

In FIGS. 13 and 14, the impact of data preprocessing/correction is highlighted. In FIG. 13, a typical experimental raw absorbance spectrum 1300 collected from the perfused pig ear based on diffuse reflectance is depicted. The spectrum contains signals from the tissue—skin, its constituents (collagen, water, etc.) and the perfused solution, which in this particular case is 2% ethanol aqueous solution. In this experiment, ethanol is the analyte of interest. The solution is injected into an artery of the ear and is collected back through the vein. The sensor is attached to the surface of the skin of the ear and collects diffuse reflectance of the tissue as well as perfused solution.

Due to the nonlinear nature of the diffuse reflectance one of the important steps in data preprocessing is the linearization and scattering correction of the collected spectrum, which, when correctly applied, allows further processing of the data for instance Beer-Lambert absorbance based analysis, where of the linearized and corrected spectrum is decomposed into individual components. This subsequent analysis may be performed in combination with other linear regression techniques to obtain a calibrated value of the concentration level of the constituent/analyte of interest.

In FIG. 13, Kubelka-Munk linearization is performed in decomposing the raw spectrum 1300 and, by using a pure ethanol absorption spectrum 1400 obtained from calibrated transmission measurements, also referred to as the reference spectrum of a selected analyte, one can isolate/decompose ethanol in the observed transdermal spectrum 1500. While noisy, as no additional processing was performed, the isolated spectrum 1500 does show three ethanol-specific peaks.

Further processing of the isolated spectrum can be performed as shown in FIGS. 14a and 14b. Here, a 24 hour perfusion cycle of different ethanol concentrations, ranging from 0.1% to 2% was performed. Control flow cuvettes at the input of the artery and at the output of the vein were used to monitor the perfused solution concentrations and its stability and is depicted as the reference cuvette signal 1600. Sensing was performed transdermally based on diffuse reflectance geometry.

In FIG. 14a, the obtained raw spectrum 1300 was processed directly by applying −ln(x) for fitting a Beer-Lambert model, without any linearizing transformation/correction. The components used for fitting included water, skin, ethanol, fat, slope, pathlengths, and an offset and, as such, the spectrum was decomposed into water, skin, fat, ethanol, slope, path lengths, and an offset. The resulting fit was compared to the control cuvette measurement, i.e., the reference cuvette signal 1600. It can be seen that while there is some correlation of the ethanol trace 1700a with the reference trend 1600, it is mostly inconclusive and does not provide reliable reading for sensing applications.

In FIG. 14b, the same diffuse reflectance spectrum was processed for linearization and scattering correction using Kubelka-Munk correction, followed by Beer-Lambert approximation (decomposition and fitting of individual components). In this case, the extracted transdermal ethanol trace 1700b agrees well with the reference cuvette signal 1600 in the entire 0.1%-2% range, including the abruptly increasing/decreasing profiles.

The described embodiments of the invention are intended to be merely exemplary and numerous variations and modifications are intended to be within the scope of the present invention as defined in the appended claims.

1. A method for calibrating a sensor for measurement of concentration of an analyte, the method comprising:
collecting, using a hybrid group III-V/group IV semiconductor photonics system-on-a-chip (SoC), a plurality of raw spectra from an object having the analyte;
partitioning the plurality of raw spectra according to respective spectral shapes thereof into a set of clusters, each cluster comprising a group of raw spectra; and
within each cluster:
applying a respective local scattering correction (LSC) to each raw spectrum belonging to the cluster to obtain a group of locally corrected spectra; and
deriving, using the locally corrected spectra and gold standard analyte concentration values corresponding to the group of raw spectra belonging to the cluster, a cluster-specific optimized set of pre-processing parameters and a cluster-specific calibration vector.

2. The method of claim 1, wherein deriving the cluster-specific optimized set of pre-processing parameters and the cluster-specific calibration vector for a particular cluster comprises:
evaluating each of a plurality of candidate sets of pre-processing parameters, evaluation of a particular candidate set comprising:
pre-processing each locally corrected spectrum belonging to the particular cluster using the particular candidate set;
deriving a candidate calibration vector by applying multivariate regression calibration to the pre-processed locally corrected spectra and using the gold-standard analyte concentration values corresponding to the group of raw spectra belonging to the particular cluster; and
computing a corresponding accuracy measure for the candidate calibration vector via cross-validation; and
designating the candidate set and the corresponding candidate calibration vector associated with a maximum accuracy measure as the cluster-specific optimized set of pre-processing parameters and cluster-specific calibration vector, respectively.

3. The method of any preceding claim, wherein:
the object comprises tissue; and
the analyte comprises at least one of: blood glucose, blood lactate, ethanol, urea, creatinine, troponin, cholesterol, albumin, globulin, ketones-acetone, acetate, hydroxybutyrate, collagen, keratin, or water.

4. The method of any preceding claim, wherein partitioning the plurality of raw spectra according to respective spectral shapes thereof comprises:
applying a global scattering correction (GSC) to each of the plurality of raw spectra to obtain a plurality of globally corrected spectra;
clustering the plurality of globally corrected spectra according to: (A) a specified number of clusters, or (B) a specified maximum distance of a globally corrected spectrum from a centroid of a cluster, or (C) both a specified number of clusters and a specified maximum distance to a globally corrected spectrum from a centroid of a cluster; and
within each cluster, designating to that cluster a respective raw spectrum corresponding to a globally corrected spectrum belonging to the cluster.

5. The method of claim 4, wherein the clustering comprises at least one of: k-means clustering, affinity propagation, or agglomerative clustering.

6. The method of any preceding claim, further comprising:
storing in the SoC a GSC reference spectrum.

7. The method of any of claim 4 or claim 5, wherein the global scattering correction comprises global multiplicative scattering correction, global standard normal variate (SNV) correction, Kubelka-Munk correction, Saunderson correction, or global mean centering and normalization correction.

8. The method of any of claim 4 or claim 5, where the local or global scattering correction comprises particle-size difference correction or pathlength-difference correction, each correction comprising Kubelka-Munk correction, Saunderson correction, multiplicative scattering correction, or a combination thereof.

9. The method of any preceding claim, further comprising:
storing in the SoC, for each cluster: (i) a corresponding LSC reference spectrum, (ii) a corresponding calibration vector, and (iii) cluster centroid.

10. The method of claim 9, further comprising:
storing in the SoC, for each cluster: (iv) the cluster-specific optimized set of pre-processing parameters.

11. The method of any preceding claim, further comprising:
storing in the SoC the optimized set of pre-processing parameters for each cluster.

12. The method of any preceding claim, wherein the local scattering correction comprises local multiplicative scattering correction, local standard normal variate (SNV) correction, Kubelka-Munk correction, Saunderson correction, or local mean centering and normalization correction.

13. The method of any preceding claim, wherein determining the respective spectral shapes of the plurality of raw spectra comprises:
pre-processing the plurality of raw spectra by applying thereto a linear transformation and a baseline correction based on a reference spectrum of a selected analyte.

14. The method of claim 13, wherein the pre-processing comprises Kubelka-Munk correction, Saunderson correction, multiplicative scattering correction, or a combination thereof.

15. A method for measuring concentration of an analyte, the method comprising:
obtaining, using a hybrid group III-V/group IV semiconductor photonics system-on-a-chip (SoC), a raw spectrum from an object having the analyte;
identifying from a plurality of clusters of spectra a cluster to which the raw spectrum belongs based on spectral shape of the raw spectrum;
applying a local scattering correction (LSC) to the raw spectrum to obtain a locally corrected spectrum;
pre-processing the locally corrected spectrum using a cluster-specific optimized set of pre-processing parameters; and
multiplying the preprocessed locally corrected spectrum with a cluster-specific calibration vector to obtain a calibrated concentration value for the analyte.

16. The method of claim 15, wherein obtaining the raw spectrum comprises:
directing from the SoC to the object electromagnetic radiation (EMR) tunable at a plurality of wavelengths;
measuring using the SoC intensities of EMR received from the object at each of the plurality of wavelengths; and
converting the intensities into absorbance values, wherein the raw spectrum comprises an absorbance spectrum.

17. The method of claim 16, wherein the plurality of wavelengths are selected from a range 1000 nm-3500 nm or a range 1900-2500 nm.

18. The method of any of claims 15 to 17, wherein:
the plurality of clusters of spectra correspond to spectra collected previously using the SoC; and
each of the plurality of clusters is represented via a respective LSC reference, cluster centroid and a respective calibration vector, the respective LSC reference, the respective cluster centroid, and the respective calibration vector for each cluster being stored on the SoC.

19. The method of any of claims 15 to 18, wherein identifying from the plurality of clusters of spectra the cluster to which the raw spectrum belongs comprises:
deriving a globally corrected spectrum using a global scattering correction (GSC) reference;
within each cluster from the plurality of clusters:
comparing the globally corrected spectrum with a respective LSC reference to obtain a distance corresponding to that cluster; and
selecting a cluster for which the corresponding distance is minimum.

20. The method of claim 19, wherein the global scattering correction comprises global multiplicative scattering correction, global standard normal variate (SNV) correction, Kubelka-Munk correction, Saunderson correction, global mean centering and normalization correction, or a combination thereof.

21. The method of claim 19, where the local or global scattering correction comprises particle-size difference correction or pathlength-difference correction such as Kubelka-Munk, Saunderson correction, multiplicative scattering correction, or a combination thereof.

22. The method of any of claims 15 to 21, wherein the local scattering correction comprises local multiplicative scattering correction, local standard normal variate (SNV) correction, or local mean centering and normalization correction, Kubelka-Munk correction, Saunderson correction, or a combination thereof.

23. The method of any of claims 15 to 22, wherein determining the spectral shape of the raw spectrum comprises:
pre-processing the raw spectrum by applying thereto a linear transformation and a baseline correction based on a reference spectrum of a selected analyte.

24. The method of claim 23, wherein the pre-processing comprises Kubelka-Munk correction, Saunderson correction, multiplicative scattering correction, or a combination thereof.

25. A system for measuring concentration of an analyte, comprising:
a hybrid group III-V/group IV semiconductor photonics system-on-a-chip (SoC) for obtaining a raw spectrum from an object having the analyte; and
a processing unit, comprising a processor and memory, and configured to:
obtain, using the hybrid group III-V/group IV semiconductor photonics system-on-a-chip (SoC), a raw spectrum from an object having the analyte;
identify from a plurality of clusters of spectra a cluster to which the raw spectrum belongs based on spectral shape of the raw spectrum;
apply a local scattering correction (LSC) to the raw spectrum to obtain a locally corrected spectrum;
preprocess the locally corrected spectrum using a cluster-specific optimized set of pre-processing parameters; and
multiply the preprocessed locally corrected spectrum with a cluster-specific calibration vector to obtain a calibrated concentration value for the analyte.

26. The system of claim 25, wherein:
to obtain the raw spectrum, the SoC is configured to:
direct to the object electromagnetic radiation (EMR) tunable at a plurality of wavelengths; and
measure intensities of EMR received from the object at each of the plurality of wavelengths; and
the processor is programmed to convert the intensities into absorbance values, wherein the raw spectrum comprises an absorbance spectrum.

27. The system of claim 26, wherein the plurality of wavelengths comprises a range 1000 nm-3500 nm or a range 1900-2500 nm.

28. The system of any of claims 25 to 27, wherein:
the plurality of clusters of spectra correspond to spectra collected previously using the SoC;
each of the plurality of clusters is represented via a respective LSC reference, a respective cluster centroid, and a respective calibration vector; and
the SoC comprises memory for storing, for each cluster, the respective LSC reference, the respective cluster centroid, and the respective calibration vector.

29. The system of any of claims 25 to 28, wherein the SoC comprises memory for storing the optimized set of pre-processing parameters for each cluster.

30. The system of any of claims 25 to 29, wherein to identify from the plurality of clusters of spectra the cluster to which the raw spectrum belongs, the processor is programmed to:
derive a globally corrected spectrum using a global scattering correction (GSC) reference;
within each cluster from the plurality of clusters:
compare the globally corrected spectrum with a respective LSC reference to obtain a distance corresponding to that cluster; and
select a cluster for which the corresponding distance is minimum.

31. The system of claim 30, wherein the global scattering correction comprises global multiplicative scattering correction, global standard normal variate (SNV) correction, Kubelka-Munk correction, Saunderson correction, or global mean centering and normalization correction.

32. The system of claim 30, where the local or global scattering correction comprises particle-size difference correction or pathlength-difference correction, each correction comprising Kubelka-Munk correction, Saunderson correction, multiplicative scattering correction, or a combination thereof.

33. The system of any of claims 25 to 32, wherein the local scattering correction comprises local multiplicative scattering correction, local standard normal variate (SNV) correction, Kubelka-Munk correction, Saunderson correction, or local mean centering and normalization correction or a combination thereof.

34. The system of any of claims 25 to 33, wherein the SoC comprises:
a wavelength shift tracker to track a shift in wavelength of radiation emitted by the SoC, a wavelength tracker to track absolute wavelength of the radiation emitted by the SoC;
a temperature sensor to measure the temperature of the SoC; and
an SoC output power monitor to monitor the intensity of the EMR emitted by the SoC during a wavelength sweep.

35. The system of any of claims 25 to 34, wherein to determine the respective spectral shapes of the plurality of raw spectra, the processing unit is configured to:
pre-process the plurality of raw spectra by applying thereto a linear transformation and a baseline correction based on a reference spectrum of a selected analyte.

36. The system of claim 35, wherein while performing the pre-processing, the processing unit is configured to apply Kubelka-Munk correction, Saunderson correction, multiplicative scattering correction, or a combination thereof.
